(19) United States
(12) Patent Application Publication
(10) Pub. No.: US 2026/0102524 A1
(43) Pub. Date: Apr. 16, 2026

(54) KIT FOR RADIOLABELLING WITH 68GA COMPRISING A METAL INHIBITOR

(71) Applicant: TELIX INNOVATIONS S.A., HERSTAL (BE)

(72) Inventors: Ludovic Wouters, Herve (BE); Geoffroy Kaisin, Seraing (BE); André Luxen, Ocquier - Clavier (BE); Marc Léonard, Flémalle (BE); Samuel Voccia, Liége (BE)

(73) Assignee: TELIX INNOVATIONS S.A., HERSTAL (BE)

(21) Appl. No.: 19/409,099

(22) Filed: Dec. 4, 2025

Related U.S. Application Data
(63) Continuation of application No. 17/742,502, filed on May 12, 2022.
Application No. 17/742,502 is a continuation of application No. 17/324,243, filed on May 19, 2021.
Application No. 17/324,243 is a continuation of application No. 15/505,941, now Pat. No. 11,040,120.

(30) Foreign Application Priority Data
Aug. 29, 2014 (BE) 2014/0653

(51) Int. Cl. A61K 51/04; C07B 59/00

(52) U.S. Cl. CPC A61K 51/0482; C07B 59/008; C07B 2200/05

(57) ABSTRACT

The present invention relates to a kit for the radiolabeling of a targeting agent with gallium-68. The present invention also relates to the use of said kit for the radiolabeling of a targeting agent, to a method for the radiolabeling of a targeting agent with gallium-68 using said kit, and to a method for the preparation of said kit.

### TECHNICAL FIELD

The present invention relates to a kit suitable for a radiolabeling reaction.

### CONTEXT

Recently, some very interesting clinical results based on molecules radiolabeled with ⁶⁸Ga for imaging in in vivo by PET have been published and presented. These radiotracers are generally an assembly of a chelating agent with a targeting agent, making possible, respectively, the reaction with a metallic isotope and the biological/metabolic activity of the radiotracer. However, due to the short half-life of ⁶⁸Ga (68 minutes), the radiotracers based on this radioisotope are not suitable for long-distance distribution and additionally require production equipment, such as automated synthesizers, for the labeling process, making difficult widespread use routinely in nuclear chemistry.

The labeling reaction with ⁶⁸Ga is carried out by chelating the radioactive metal with an appropriate chelating agent in an appropriate reaction medium, generally in a medium buffered in order to ensure an optimum pH for the chelation reaction.

⁶⁸Ga is obtained from a generator. Said generator is an alternative to the in situ production using a cyclotron or to the daily delivery of radioisotopes. The system was initially developed for ⁹⁹Tc. The principle is based on the radiochemical separation between a parent element of long half-life (or nonradioactive element, such as ⁶⁸Ge) contained in the generator and a daughter element of short half-life resulting from the disintegration of the parent element. The daughter is recovered with an excellent radiochemical and radionuclide purity (that is to say, without contamination by other radionuclides or other radiochemical impurities) and with a good chemical purity (low content of metal ions). The separation is rendered possible by virtue of the different chemical properties of the two elements (parents and daughters).

The characteristics of the ⁶⁸Ge/⁶⁸Ga generator can be summarized as follows:
The eluate is obtained in an acid solution (0.05M-1M in HCl, specified by the manufacturer of the generator)
The eluate contains ⁶⁸Zn, resulting both from the manufacture of the ⁶⁸Ge and from the disintegration of the ⁶⁸Ga, the concentration of which continually increases and depends on the time elapsed since the last elution of the generator. This ⁶⁸Zn indeed accumulates in the generator. This can be detrimental to the radiolabeling yield insofar as this ⁶⁸Zn competes directly with the ⁶⁸Ga in the chelation reactions used for the radiolabeling.
The eluate contains ⁶⁸Ge (the “breakthrough”).
The eluate also contains a variety of leaching metals originating from the solid phase of the generator column, from the pipes but also from the HCl used for the elution:
   Microg/ml level: Fe(III), Zn(II), Al(III)
   Picog/l level: Mn(II), Pb(II), Ti(IV), Cr(III), Ni(II) (Sn(IV))
      The chelation reaction is dependent on an appropriate pH but also on the possible competition of the abovementioned metal impurities with the ⁶⁸Ga during the chelation reaction. In addition, it is generally accepted that heat promotes the chelation reaction for the most commonly used radiotracers based on ⁶⁸Ga.

In the state of the art, the presence of metal ions which compete with the ⁶⁸Ga is generally reduced by a prelabeling purification or the fractionation of the eluate (as described in the document WO 2010/092114), but these stages represent a loss of starting activity resulting either from the time lost or from the process itself.

The possibility of a partial chelation of the ⁶⁸Ga requires, in general, the final postlabeling purification which makes it possible to obtain a radiotracer having a radiochemical purity which meets pharmaceutical specifications (radiochemical purity>90%). These stages also represent a loss of activity resulting from the time lost or from the process itself.

According to known methods, at the end of the labeling, a sequestering agent having a particular affinity for gallium can be added in order to chelate the unreacted part of the isotope. In addition, the need for these pre-and postlabeling purification stages makes the syntheses of radiotracers labeled with ⁶⁸Ga dependent, to a certain extent, on automatization and the use of a synthesis module. Besides the necessary technical expertise, this requires additional time unfavorable to the overall yield.

Due to the short half-life of the radionuclide (68 minutes) and to the limited activity provided by the generator, any improvement for the purpose of obtaining a rapid, direct and high-yield chelation is very desirable.

The desired buffer should be non-toxic, be capable of effectively keeping the pH in a range from 3.0 to 5.0, should not compete with the gallium ions and should preferably have a low ability to chelate metals. It should also be capable of tolerating the possible small variations in the volume of generator eluate (and thus in the amount of HCl), that is say that it should be sufficiently strong to keep the pH in the desired range with variations of 10% in the eluate volume.

The management of the competing metal impurities is another challenge. It has been shown, in WO2013024013, that the addition of a cochelating agent can make it possible to inhibit competing metal impurities. This is because any entity which would make it possible to inhibit metal impurities while avoiding negatively interfering, or having a limited ability to negatively interfere, with the gallium chelation reaction can act as a trap for these impurities. In other words, this inhibiting effect brings the apparent concentration of competing metal, that is to say the concentration of metal impurities still available for the chelation, to a level which makes possible high and reproducible radiolabeling yields.

It is thus clear that there exists a need for an improved method for the preparation of 68Ga chelates which overcomes one or more of the abovementioned problems. This involves the identification of an appropriate medium which makes it possible to keep the pH within a tolerable range, to manage the metal contamination, which avoids the need to heat in order to promote the chelation reaction and which makes possible chelation yields of the 68Ga of greater than 90%.

### SUMMARY

The present invention relates to a kit for the radiolabeling of a targeting agent with gallium-68, said kit comprising:
an appropriate amount of acetate salt which makes it possible to balance at least the acid pH of an eluate of a gallium-68 generator at a pH value ranging from 3 to 5 when said generator is eluted in the kit; and
a metal-inhibiting agent capable of inactivating the metals other than gallium-68 and without negatively interfering with the chelation of the gallium-68, under the conditions of the labeling reaction,
in which the acetate salt and the metal-inhibiting agent are (co)lyophilized.

Another subject matter of the invention is the use of a kit according to the invention for the radiolabeling of a targeting agent, in particular for the radiolabeling of a targeting agent with gallium-68.

The invention additionally provides a method for the radiolabeling of a targeting agent with gallium-68, comprising the elution of a gallium-68 generator with an eluent comprising an acid, in a kit according to the invention.

The invention also relates to a solution which can be obtained by elution of a gallium-68 generator with an eluent comprising an acid, in a kit according to the invention.

The invention additionally provides a method for the preparation of a kit according to the invention, comprising the following stages:
a) preparation of a solution comprising the acetate salt and a metal-inhibiting agent, and optionally a targeting agent functionalized by a chelating agent; and
b) the lyophilization of the solution obtained in stage a).

### DETAILED DESCRIPTION

As used here, the singular forms “a”, “an” and “the” comprise the singular and plural referents, unless the context clearly indicates the contrary.

The terms “comprising”, “comprises” and “composed of” as used here are synonyms of “including”, “comprises” or “containing”, “contains”, and are inclusive or open and do not exclude additional unmentioned elements or stages of the method. The terms also encompass “consisting of” and “consisting essentially of”, which benefit from meanings well established in patent terminology.

The recitation of the numerical ranges as parameter comprises all the numbers and the fractions encompassed within the respective ranges, and also the evaluation criteria cited.

The terms “approximately” and “roughly” as used here in reference to a measurable value, such as a parameter, an amount, a temporal duration and the like, are intended to encompass variants of and from the specified value, such as variations of +/−10% or less, preferably +/−5% or less, preferably +/−1% or less, and more preferably still +/−0.1% or less of and from the specified value, insofar as these variations are appropriate for being used in the invention disclosed. It should be understood that the value to which the modifier “approximately” or “roughly” refers is itself also specifically, and preferably, disclosed.

Unless otherwise defined, all the terms used in the disclosure of the invention, including the technical and scientific terms, have the meanings commonly understood by a person skilled in the art to which this invention belongs. Through further information, definitions of terms are included in order to better appreciate the teaching of the invention. When specific terms are defined in the context of a particular aspect of the invention or a particular embodiment of the invention, such connotation is intended to apply throughout this description, namely also in the context of other aspects or embodiments of the invention, unless otherwise indicated.

In the following passages, different aspects or embodiments of the invention are defined in more detail. Each aspect or embodiment thus defined can be combined with each of the other aspects or embodiments, unless otherwise mentioned. In particular, any function indicated as being preferred or advantageous can be twinned with any other entity or entities indicated as being preferred or advantageous.

The present invention overcomes one or more of the problems stated and observed above and makes it possible, in the context of the radiolabeling of the targeting agent with gallium-68 using a kit as described here, this gallium-68 being eluted from a ⁶⁸Ge/⁶⁸Ga generator, to effectively radiolabel the targeting agent functionalized by a chelating agent in a buffered aqueous solution.

Consequently, in one aspect, the invention provides a kit comprising:
an amount of acetate salt appropriate for balancing at least the acid pH of an eluate from a gallium-68 generator at a pH value ranging from 3 to 5 when said generator is eluted in said kit; and
a metal-inhibiting agent capable of rendering inactive the metals other than gallium-68 without negatively interfering with the chelation of the gallium-68, under the conditions of the labeling reaction,
in which the acetate salt and the metal-inhibiting agent are (co)lyophilized.

The kit as described in this document makes it possible not only to establish an optimum pH for carrying out the chelation or radiolabeling reaction but also to tolerate the variation in the volume of eluate and in acidity associated with different types of gallium-68 generators, through the use of an appropriate amount of acetate salt which, when it is mixed with the acidic generator eluate, forms an acetic acid/acetate buffer having a pH in the range 3-5. In this way, the amount of nonchelated gallium-68 due to an excessively low or excessively high pH, which results in a high content of free ⁶⁸Ga³⁺ or ⁶⁸Ga hydroxides (colloidal gallium) respectively, is reduced.

In addition, the acetate buffer is well tolerated as buffer or as excipient for pharmaceuticals.

In preferred embodiments, the radiolabeling reaction is carried out at a temperature close to or equal to ambient temperature, preferably at a temperature of less than 50° C. and more preferably at ambient temperature.

Furthermore, the present inventors have found that a metal-inhibiting agent can be used in the radiolabeling method in order to neutralize, at least in part, the interfering entities, leaving the ⁶⁸Ga free to react with the targeting agent functionalized by a chelating agent. These metal-inhibiting agents can, temporarily or permanently, remove the metals in competition for the reaction with targeting agents functionalized by a chelating agent. The presence of metal-inhibiting agents during the radiolabeling reaction provides an advantageous alternative to current strategies for managing the presence of metal impurities, such as the increase in the amount of targeting agent functionalized by a chelating agent or the pretreatment of the eluate from the generator, these additional purification stages consuming time (and radioactivity).

These aspects advantageously make it possible to obtain an appropriate degree of chelation, in particular of at least 90%, and consequently a sufficient purity without any prior or final purification.

In preferred embodiments, the kit additionally comprises a targeting agent functionalized by a chelating agent and lyophilized.

The presence of a targeting agent functionalized by a chelating agent, an acetate salt and a metal-inhibiting agent in the kit advantageously makes it possible to directly elute a gallium-68 generator in the kit and carry out the radiolabeling reaction, without the need for any prior or subsequent manipulation.

In addition, all of the kit components as described here are lyophilized, which guarantees a longer shelf life.

Thus, the main advantages of a kit as described here, which differentiate said kit from the kits of the state of the art, are:
A completely dry kit which makes possible a better lifetime of the targeting agent functionalized by a chelating agent.
The possibility of radiolabeling without the need for an automated synthesizer.
The possibility of radiolabeling without the need for heating.
The presence of a metal-inhibiting agent which advantageously makes it possible to use less targeting agent functionalized by a chelating agent and making possible the implementation of syntheses of cheaper radiopharmaceuticals.
Any brand of generator can be used with this kit provided that the acetate is partially neutralized with HCl so that, when it is mixed with the acid eluate from the generator, the optimum pH for radiolabeling is obtained.

As used here, “acetate” refers to the anionic CH₃COO⁻ molecule. “Acetate salt” is understood here to mean any metal salt of the acetate. Nonlimiting examples of acetate salts comprise sodium acetate, potassium acetate, aluminum acetate and ammonium acetate. Preferably, sodium acetate is used in the kits, as described here.

The amount of acetate salt present in the kit as described here can be adjusted as a function of the type and/or the brand of the gallium-68 generator; in particular, the amount of acetate salt present in the kit is capable of balancing the pH of a certain amount of HCl originating from a generator eluate so that the resulting solution exhibits a pH of between 3 and 5, preferably between 3.5 and 4.5, preferably between 3.9 and 4.3.

Alternatively, the kit as described in the present invention can comprise a fixed amount of acetate salt. The differences in amount of HCl originating from the generator eluate (as a function of the type and/or the brand of gallium-68 generator) can then be adjusted by adding an appropriate amount of HCl to the kit as described in the present invention before the elution. The amount of HCl to be added to the kit as described in the present invention must partially neutralize the acetate salt so that the non-neutralized acetate salt is capable of balancing the pH of a certain amount of HCl originating from a generator eluate so that the resulting solution exhibits a pH of between 3 and 5, preferably between 3.5 and 4.5, preferably between 3.9 and 4.3.

Preferably, the acetate salt is present in the kit as taught here in an amount of between approximately 5 mg and approximately 1000 mg, preferably in an amount of between approximately 25 mg and approximately 750 mg, more preferably in an amount of between approximately 100 mg and approximately 500 mg.

The metal-inhibiting agents used in the present invention are chosen for their ability to inhibit the competing metals without, however, inhibiting the 68Ga ions in their chelation reaction with the targeting agent functionalized by a chelating agent. This is because these metal-inhibiting agents must not negatively interfere with the main radiolabeling reaction or result in the formation of secondary radiolabeled entities.

It is interesting to note that the function of the metal-inhibiting agents in the present invention is the opposite of the function of the sequestering agents used in the prior art. This is because, according to the known methods, at the end of the labeling reaction, a sequestering agent having a particular affinity for gallium can be added in order to chelate the unreacted part of the isotope, whereas, according to the present invention, an agent capable of reducing the competition of metal impurities inhibiting metals other than gallium-68 is added at the start of the reaction.

Furthermore, the fact of being able to carry out the radiolabeling reaction at a temperature close to ambient temperature (<50° C.) advantageously makes it possible to use metal-inhibiting agents which would not be usable at the usual temperatures for the radiolabeling of targeting agents functionalized by DOTA as used in WO2013024013, because they would directly compete with gallium-68 at these temperatures. The temperature is thus also in the invention described here a lever making it possible to regulate the reactivity of the metal-inhibiting agent.

As used here, a “metal-inhibiting agent” refers to any molecule capable, by its interaction with either the competing metals, or the chelating part of the targeting agent functionalized by a chelating agent, or with gallium-68 directly, of inhibiting, in all or in part, the chelation by the targeting agent functionalized by a chelating agent of said competing metals and/or of promoting the chelation of gallium-68 by said targeting agent.

The metal-inhibiting agents are preferably chosen from the group comprising or consisting of: DOTA and its derivatives, such as TRITA, DO3A-Nprop, BisDO3A and TrisDO3A; DTPA and its derivatives, such as tetra-tBu-DTPA, p-SCN-Bz-DTPA, MX-DTPA and CHX-DTPA; and sugars. The sugars used as metal-inhibiting agents in the kit according to the invention can be monosaccharides or derivatives of monosaccharides, such as tetraketose, pentaketose, hexaketose, tetrose, pentose, hexose, D-mannose, D-fructose and their derivatives; and/or disaccharides and their derivatives, such as maltose and its derivatives; and/or polysaccharides and their derivatives, such as dextrins, cyclodextrins, cellulose and their derivatives.

Preferably, the metal-inhibiting agent is present in the kit as described here in micromolar amounts, preferably in nanomolar amounts, preferably in an amount of less than 500 nanomoles, more preferably still in an amount of less than 100 nanomoles.

It is important to note that the metal-inhibiting agents as indicated above can also advantageously be used in chelation reactions in which buffers other than acetic acid/acetate buffers are used.

The metal-inhibiting agents as indicated above can also advantageously be used in chelation reactions in which said metal-inhibiting agent is included in the eluent of the generator or in the HCl solution or in the water possibly added before the elution of the generator and is thus found in the radiolabeling solution. The metal-inhibiting agent can also be chemically bonded by a bond which is labile under the conditions of radiolabeling with the targeting agent functionalized by a chelating agent, in such a way that, under the radiolabeling conditions, the metal-inhibiting agent is formed and released in situ.

As used here, a “targeting agent functionalized by a chelating agent” refers to a targeting agent capable of being labeled with a radioisotope, such as, for example, gallium-68, via a chelating agent to which this molecule is bonded.

Preferred chelating agents for functionalizing a targeting agent to be radiolabeled with 68Ga are those which form with Ga³⁺, in particular ⁶⁸Ga³⁺ (the radioisotope eluted from a ⁶⁸Ge/⁶⁸Ga generator using HCl), chelates stable at least for a sufficient time for diagnostic examinations using radiolabeled targeting agents. Appropriate chelating agents include linear or macrocyclic aliphatic amines, such as macrocyclic amines with tertiary amines. Although these examples of appropriate chelating agents are nonlimiting, they preferably comprise NOTA and its derivatives, such as TACN, TACN-TM, DTAC, H3NOKA, NODASA, NODAGA, NOTP, NOTPME, PrP9, TRAP, TRAP-Pr, NOPO, TETA; open-chain chelates, such as HBED, MPO, EDTA, 6SS, B6SS, PLED, TAME, YM103; NTP (PRHP) 3; H2dedpa and its derivatives, such as H2dedpa-1, 2-H2dedpa, H2dp-bb-NCS, and H2dp-N-NCS; (4,6-MeO2sal) 2-BAPEN; and citrate and its derivatives.

The targeting agent functionalized by a chelating agent can be a peptide, for example a peptide comprising from 2 to 20 amino acids, a polypeptide, a protein, a vitamin, a saccharide, for example a monosaccharide or a polysaccharide, an antibody, a nucleic acid, an aptamer, an antisense oligonucleotide or an organic molecule.

The targeting agents functionalized by a chelating agent as described here preferably have a biological targeting capacity. Nonlimiting examples of appropriate targeting agents comprise molecules targeting VEGF receptors, bombesin analogs or molecules targeting GRP receptors, molecules targeting somatostatin receptors, RGD peptides or molecules targeting avß3 and avß5, annexin V or molecules targeting apoptotic processes, molecules targeting estrogen receptors, biomolecules targeting atheroma plaque, and the like. More generally, a list of biological or nonbiological targeting molecules functionalized by a chelating agent can be found in the review by Velikyan et al., Theranostic, 2014, Vol. 4, Number 1, “Prospective of ⁶⁸ Ga-Radiopharmaceutical Development”.

In certain embodiments, the metal-inhibiting agent and the targeting agent functionalized by a chelating agent are chemically bonded by a bond which is labile under the radiolabeling conditions, in such a way that, under the radiolabeling conditions, the metal-inhibiting agent is formed and released in situ.

In certain embodiments, the metal-inhibiting agent is included in the eluent of the generator or in the HCl solution or in the water possibly added before the elution of the generator and is thus found in the radiolabeling solution.

The various components of the kit as described here are preferably present in a container or flask, preferably a siliconized glass flask. However, this document is also targeted at a kit, in which the various components are present in separate containers or flasks.

The invention additionally provides a method for the radiolabeling of a targeting agent with gallium-68, said method comprising the elution of a gallium-68 generator with an eluent comprising an acid, in a kit as described here.

As indicated above, when the targeting agent functionalized by a chelating agent is included in the kit, the gallium-68 generator can be eluted directly in the kit. In other embodiments, the targeting agent functionalized by a chelating agent is added to a kit comprising the acetate salt and a metal-inhibiting agent as described here, before the elution.

In some embodiments, the gallium-68 generator is eluted directly in the kit. In other embodiments, water is added to the solution before the elution.

In some embodiments of the present invention, an appropriate amount of HCl is added to the solution before the elution. Said HCl is added in order to partially neutralize the acetate. The amount of HCl to be added preferably partially neutralizes the amount of acetate salt in a way such that the remaining amount of acetate salt, that is to say the amount not neutralized, is capable of balancing the pH of a certain amount of HCl originating from a generator eluate (and thus dedicated to a given type or brand of generator) so that the pH of the solution obtained for the radiolabeling or chelating reaction resulting from the addition of HCl and of the generator eluate in the kit as described here is within a pH range of between 3 and 5, preferably between 3.5 and 4.5, preferably between 3.9 and 4.3. Said HCl can be added directly to the solution, or after a certain amount of water is added to said kit.

Any gallium-68 generator can be used in the methods of the present invention. Typically, a commercial gallium-68 generator comprises a column to which germanium-68 is attached. A gallium-68 generator is typically eluted with an eluent comprising an acid, preferably HCl. Consequently, in preferred embodiments of the method, as taught here, the gallium-68 generator is eluted with an eluent comprising HCl.

After the elution of the gallium-68 generator in the kit as described here, the solution obtained is left for the radiolabeling reaction for a short period of time, in particular between approximately 2 minutes and approximately 60 minutes, preferably between approximately 2 minutes and approximately 30 minutes, for example approximately 10 minutes.

Preferably, the radiolabeling or chelation reaction is carried out at a temperature of less than 50° C., preferably at ambient temperature.

Preferably, the radiolabeling or chelation reaction is carried out at a pH of between approximately 3 and approximately 5, more preferably between approximately 3.5 and approximately 4.5, more preferably between approximately 3.9 and approximately 4.3.

This document also describes a solution obtained by elution of a gallium-68 generator with an eluent comprising an acid, preferably HCl, in a kit as taught here.

Preferably, said solution has a pH of between approximately 3 and approximately 5, preferably between approximately 3.5 and approximately 4.5, more preferably between approximately 3.9 and approximately 4.3.

The invention also describes a targeting agent radiolabeled with gallium-68, obtained by a method as described here.

In one of its aspects, the invention also provides a method for the preparation of a kit as described here, said method comprising the following stages:
a) preparation of a solution comprising the acetate salt and a metal-inhibiting agent, and optionally a targeting agent functionalized by a chelating agent; and
b) the lyophilization of the solution obtained in stage a).

The invention also relates to methods for the preparation of a kit as described here, in which the different components are lyophilized separately, as well as to methods in which the solutions to be lyophilized are prepared separately. Consequently, the present document also describes a method for the preparation of a kit as described here, comprising the following stages:
a) preparation of a solution comprising the acetate salt and lyophilization of said solution;
b) the preparation of a solution comprising a chelating agent and colyophilization of said solution;
c) optionally the preparation of a solution comprising a functionalized targeting agent chelate and lyophilization of said solution; and
d) optionally mixing the lyophilisates obtained in a), b) and optionally c).

The invention also refers to a method for the preparation of a kit as described here, comprising the following stages:
a) preparation of a solution comprising an acetate salt;
b) preparation of a solution comprising a metal-inhibiting agent;
c) optionally the preparation of a solution comprising a functionalized targeting agent chelate;
d) mixing the solutions obtained in a), b) and optionally c); and
e) lyophilization of the solution obtained in d).

### EXAMPLES

### Example 1: E&Z Generator/NODOGA-peptide Without Metal-inhibiting Agent

Labeling of a peptide with ⁶⁸Ga with an eluate of 5 ml of 0.1M HCl A commercial generator of 1850 MBq (Eckert & Ziegler) is eluted with 5 ml of 0.1M HCl (Ultrapure grade) directly in a flask containing 150 mg of lyophilized sodium acetate (Ultrapure grade), 240 μl of 3M HCl (Ultrapure grade), 760 μl of Milli-Q water and 50 μg of lyophilized NODAGA-NOC. The flask is left at ambient temperature for 10 min. The product is obtained with a radiochemical purity of 64% according to the TLC analysis of the reaction medium.

### Example 2: ITG Generator/NODOGA-peptide Without Metal-inhibiting Agent With a Non-pre-eluted Generator

Labeling of a peptide with ⁶⁸Ga with an eluate of 4 ml of 0.05M HCl

A commercial generator of 1021 MBq (ITG), unused for a few days, is eluted, without prior rinsing, with 4 ml of 0.05M HCl (Ultrapure grade) directly in a flask containing 150 mg of lyophilized sodium acetate (Ultrapure grade), 330 uμof 3M HCl (Ultrapure grade), 1670 ul of Milli-Q water and 50 μg of lyophilized NODAGA-RGD. The flask is left at ambient temperature for 10 min. The product is obtained with a radiochemical purity of 46% according to the TLC analysis of the reaction medium.

### Example 3: ITG Generator/NODOGA-peptide in the Presence of a Metal-inhibiting Agent

Labeling of a peptide with ⁶⁸Ga with an eluate of 4 ml of 0.05M HCl

A commercial generator of 1021 MBq (ITG) is eluted with 4 ml of 0.05M HCl (Ultrapure grade) directly in a flask containing 150 mg of sodium acetate (Ultrapure grade), 330 μl of lyophilized 3M HCl (Ultrapure grade), 1670 μl of Milli-Q water, 10 μg of lyophilized DOTA and 50 μg of lyophilized NODAGA-NOC. The flask is left at ambient temperature for 10 min. The product is obtained with a radiochemical purity of 93% according to the TLC analysis of the reaction medium.

### Example 4: E&Z Generator/NODOGA-peptide in the Presence of a Metal-inhibiting Agent

Labeling of a peptide with 68Ga with an eluate of 5 ml of 0.1M HCl

A commercial generator of 1850 MBq (Eckert & Ziegler) is eluted with 5 ml of 0.1M HCl (Ultrapure grade) directly in a flask containing 150 mg of lyophilized sodium acetate (Ultrapure grade), 240 μl of 3M HCl (Ultrapure grade), 760 μl of Milli-Q water, 10 μg of lyophilized D-mannose and 50 μg of lyophilized NODAGA-RGD. The flask is left at ambient temperature for 10 min. The product is obtained with a radiochemical purity of 97% according to the TLC analysis of the reaction medium.

### Example 5: E&Z Generator/NODOGA-peptide in the Presence of a Metal-inhibiting Agent With a Non-pre-eluted Generator

Labeling of a peptide With ⁶⁸Ga with an eluate of 5 ml of 0.1M HCl

A commercial generator of 1850 MBq (Eckert & Ziegler), unused for a few days, is eluted, without prior rinsing, with 5 ml of 0.1M HCl (Ultrapure grade) directly in a flask containing 150 mg of lyophilized sodium acetate (Ultrapure grade), 240 μl of 3M HCl (Ultrapure grade), 760 μl of Milli-Q water, 10 μg of lyophilized DOTA and 50 μg of lyophilized NODAGA-NOC. The flask is left at ambient temperature for 10 min. The product is obtained with a radiochemical purity of 91% according to the TLC analysis of the reaction medium.

### Example 6: E&Z Generator/NODOGA-peptide in the Presence of a Metal-inhibiting Agent

Labeling of a peptide with ⁶⁸Ga with an eluate of 5 ml of 0.1M HCl

A commercial generator of 1850 MBq (Eckert & Ziegler) is eluted with 5 ml of 0.1M HCl (Ultrapure grade) directly in a flask containing 150 mg of lyophilized sodium acetate (Ultrapure grade), 240 μl of 3M HCl (Ultrapure grade), 760 μl of Milli-Q water, 10 μg of lyophilized DOTA and 50 μg of lyophilized NODAGA-NOC. The flask is left at ambient temperature for 10 min. The product is obtained with a radiochemical purity of 95% according to the TLC analysis of the reaction medium.

### Example 7: iThemba Generator/NODOGA-peptide in the Presence of a Metal-inhibiting Agent

Labeling of a peptide with ⁶⁸Ga with an eluate of 6 ml of 0.6M HCl

A commercial generator of 370 MBq (iThemba) is eluted with 6 ml of 0.6M HCl (Ultrapure grade) directly in a flask containing 421 mg of lyophilized sodium acetate (Ultrapure grade), 10 μg of lyophilized D-fructose and 50 μg of lyophilized NODAGA-NOC. The flask is left at ambient temperature for 10 min. The product is obtained with a radiochemical purity of 91% according to the TLC analysis of the reaction medium.

### Example 8: E&Z Generator/NODOGA-peptide in the Presence of a Metal-inhibiting Agent

Labeling of a peptide with ⁶⁸Ga with an eluate of 5 ml of 0.1M HCl

A commercial generator of 1850 MBq (Eckert & Ziegler) is eluted with 5 ml of 0.1M HCl (Ultrapure grade) directly in a flask containing 150 mg of lyophilized sodium acetate (Ultrapure grade), 240 μl of 3M HCl (Ultrapure grade), 760 μl of Milli-Q water, 10 μg of lyophilized D-fructose and 50 μg of lyophilized NODAGA-NOC. The flask is left at ambient temperature for 10 min. The product is obtained with a radiochemical purity of 95% according to the TLC analysis of the reaction medium.

### Example 9: ITG Generator/HBED-DUPA in the Presence of a Metal-inhibiting Agent

Labeling of an organic molecule with ⁶⁸Ga with an eluate of 4 ml of 0.05M HCl

A commercial generator of 1021 MBq (ITG) is eluted with 4 ml of 0.05M HCl (Ultrapure grade) directly in a flask containing 150 mg of lyophilized sodium acetate (Ultrapure grade), 330 μl of 3M HCl (Ultrapure grade), 1670 μl of Milli-Q water, 10 μg of lyophilized DOTA and 20 μg of lyophilized HBED-DUPA. The flask is left at ambient temperature for 10 min. The product is obtained with a radiochemical purity of 92% according to the TLC analysis of the reaction medium.

### Example 10: ITG Generator/NODOGA-peptide in the Presence of a Metal-inhibiting Agent With a Non-pre-eluted Generator

Labeling of a peptide with ⁶⁸Ga with an eluate of 4 ml of 0.05M HCl

A commercial generator of 1021 MBq (ITG), unused for a few days, is eluted, without prior rinsing, with 4 ml of 0.05M HCl (Ultrapure grade) directly in a flask containing 150 mg of lyophilized sodium acetate (Ultrapure grade), 330 μl of 3M HCl (Ultrapure grade), 1670 μl of Milli-Q water, 10 μg of lyophilized D-mannose and 50 μg of lyophilized NODAGA-NOC. The flask is left at ambient temperature for 10 min. The product is obtained with a radiochemical purity of 93% according to the TLC analysis of the reaction medium.

### Example 11: ITG generator/NODAGA-peptide in the Presence of a Metal-inhibiting Agent and 25 μg of NODAGA-peptide

Labeling of a peptide with 68Ga with an eluate of 4 ml of 0.05M HCl

A commercial generator of 1021 MBq (ITG) is eluted with 4 ml of 0.05M HCl (Ultrapure grade) directly in a flask containing 150 mg of lyophilized sodium acetate (Ultrapure grade), 330 μl of 3M HCl (Ultrapure grade), 1670 μl of Milli-Q water, 10 μg of lyophilized D-mannose and 25 μg of lyophilized NODAGA-NOC. The flask is left at ambient temperature for 10 min. The product is obtained with a radiochemical purity of 95% according to the TLC analysis of the reaction medium.

1-29. (canceled)

30. A kit for radiolabelling an HBED-functionalized targeting agent with gallium-68, said kit comprising:
an acetate salt or acetate buffer in an amount sufficient to balance an acidic pH from a gallium-68 generator when said generator is eluted in the kit; and
an HBED-functionalized targeting agent that chelates gallium-68 in radiolabelling conditions, wherein said acetate salt or acetate buffer and HBED-functionalized targeting agent are co-lyophilized in a single vial;
wherein said kit allows for the direct elution of gallium-68 from a generator with an eluent comprising HCl into said kit; and
wherein said kit allows the HBED-functionalized targeting agent to be labelled with gallium-68 in radiolabelling conditions at a temperature between 20° C. and 30° C., for a time from about 2 minutes to about 10 minutes.

31. The kit according to claim 30, wherein said targeting agent is a peptide comprising from 2 to 20 amino acids.

32. The kit according to claim 30, which allows gallium-68 chelation yields above 90%.

33. The kit according to claim 30, wherein the acetate salt or buffer is present in a fixed concentration and/or a specific amount.

34. The kit according to claim 30, wherein the buffer is a sodium acetate buffer.

35. The kit according to claim 30, wherein the acetate salt or buffer is present in an amount of between 20 and 500 mg.

36. The kit according to claim 30, wherein the acetate salt or buffer is present in an amount sufficient to balance at least the acidic pH eluate from a gallium-68 generator to a pH value ranging from about 3 to about 6 when said generator is eluted in the kit.
